(19) United States
(12) Patent Application Publication
(10) Pub. No.: US 2023/0103936 A1
(43) Pub. Date: Apr. 6, 2023

(54) PREPARATION OF S-BEFLUBUTAMID BY RESOLVING 2-(4-FLUORO-3-(TRIFLUOROMETHYL)PHENOXY)BUTANOIC ACID

(71) Applicant: Cheminova A/S, Philadelphia (US)

(72) Inventors: Ravindra V. DATAR, Bangalore (IN); Jianhua MAO, Shanghai (CN); Shaileshkumar K. PATEL, Bharuch-East (IN)

(21) Appl. No.: 17/798,858

(22) PCT Filed: Feb. 10, 2021
(86) PCT No.: PCT/IB2021/000079

§ 371 Date: Aug. 10, 2022

Related U.S. Application Data
(60) Provisional application No. 62/972,795, filed on Feb. 11, 2020.

(51) Int. Cl. C07C 231/02

(52) U.S. Cl. CPC C07C 231/02

(57) ABSTRACT

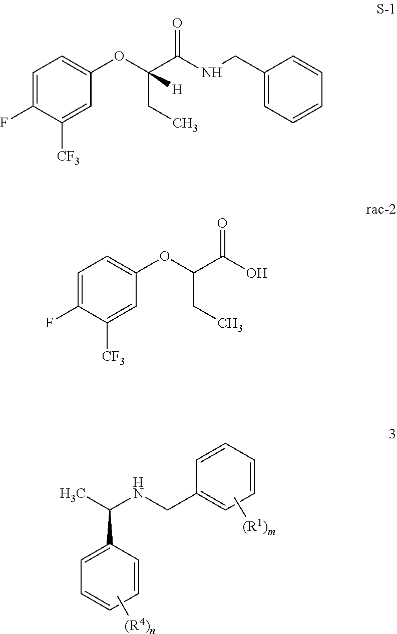

Disclosed is a method for preparing compound Formula S-1, comprising resolving compound Formula rac-2 with a compound of Formula 3 wherein R¹·R⁴, m and n are as defined in the disclosure.

### FIELD OF THE INVENTION

This invention relates to a method for preparing the S-enantiomer of beflubutamid.

### BACKGROUND OF THE INVENTION

U.S. Pat. No. 4,929,273 discloses N-benzyl-2-(4-fluoro-3-trifluoromethylphenoxy)-butanoic amide of Formula 1 as an herbicidal compound. It has a single asymmetric center at the 2-carbon of the amide moiety and thus can be a chiral molecule.

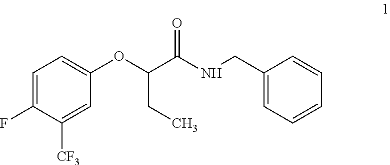 [SMILES: CCC(Oc1ccc(F)c(C(F)(F)F)c1)C(=O)NCc1ccccc1]

This compound in racemic form has been marketed commercially under the common name beflubutamid as a soil herbicide for pre- and post-emergence control of dicotyledonous weeds in cereals. It inhibits the enzyme phytoene-desaturase that is involved in the biosynthesis of carotenoids. Depletion of carotenoids leads to photooxidation of chlorophyll and bleaching/chlorosis of susceptible weeds.

U.S. Pat. No. 4,929,273 also discloses that the (−)-optical isomer is more herbicidally active than the racemic mixture. The more active enantiomer has been identified as having the S-configuration shown as compound S-1 (Environ. Sci. Technol. 2013, 47, 6806-6811 and Environ. Sci. Technol. 2013, 47, 6812-6818).

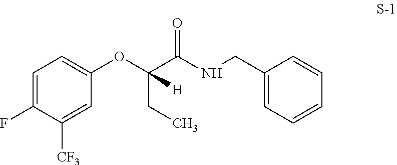 [SMILES: CC[C@H](Oc1ccc(F)c(C(F)(F)F)c1)C(=O)NCc1ccccc1]

While the methods disclosed in the preceding reference can provide the desired compound of Formula S-1, continuous improvements are sought, particularly in the development of methods to provide materials on a commercial scale. Therefore, the need continues for new methods that are less costly, more efficient, more flexible, or more convenient to operate.

### SUMMARY OF THE INVENTION

Embodiment A. This invention provides a method for preparing compound S-1

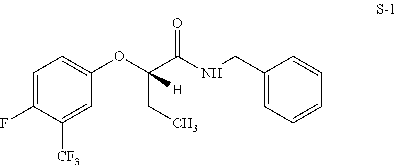 [SMILES: CC[C@H](Oc1ccc(F)c(C(F)(F)F)c1)C(=O)NCc1ccccc1]

from compound S-2

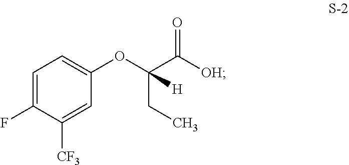 [SMILES: CC[C@H](Oc1ccc(F)c(C(F)(F)F)c1)C(=O)O]

wherein compound S-2 is prepared by treating compound rac-2

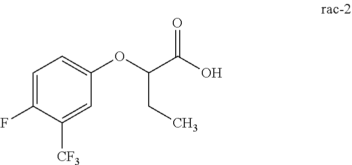 [SMILES: CCC(Oc1ccc(F)c(C(F)(F)F)c1)C(=O)O]

with a compound of Formula 3

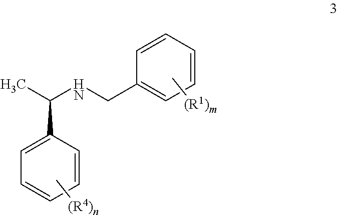

wherein
each R¹ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R²; or
two adjacent R¹ substituents are taken together with the phenyl to which they are attached to form a naphthalenyl ring optionally substituted with up to three R³;
each R² and each R³ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
each R⁴ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R⁵;
each R⁵ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
m is 0, 1, 2 or 3; and
n is 0, 1, 2 or 3;

to provide a R,S-salt of Formula 4

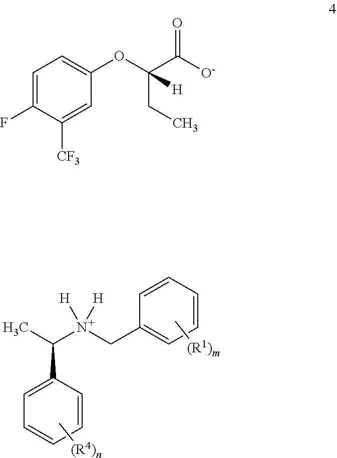

wherein R¹, R⁴, m and n are as defined above;

selectively isolating the R,S-salt of Formula 4;

treating the R,S-salt of Formula 4 with a sodium base to provide the carboxylate salt S-5

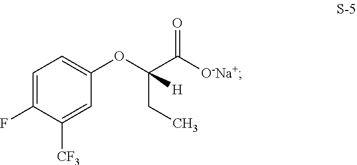

treating the carboxylate salt S-5 with acid.

Embodiment B. This invention also provides a method for preparing compound S-1

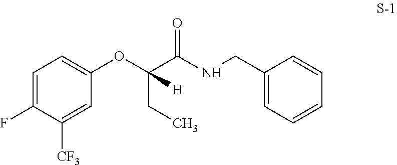 [SMILES: CC[C@H](Oc1ccc(F)c(C(F)(F)F)c1)C(=O)NCc1ccccc1]

the method comprising:

treating compound rac-2

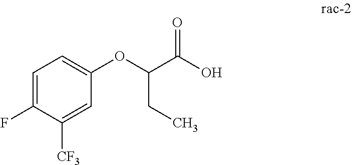 [SMILES: CCC(Oc1ccc(F)c(C(F)(F)F)c1)C(=O)O]

with a compound of Formula 3

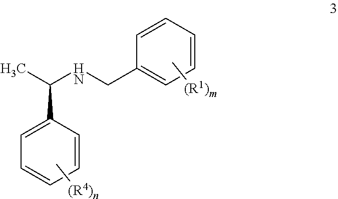

wherein
each R¹ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R²; or
two adjacent R¹ substituents are taken together with the phenyl to which they are attached to form a naphthalenyl ring optionally substituted with up to three R³;
each R² and each R³ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
each R⁴ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R⁵;
each R⁵ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
m is 0, 1, 2 or 3; and
n is 0,1,2 or 3;

to provide the R,S-salt of Formula 4

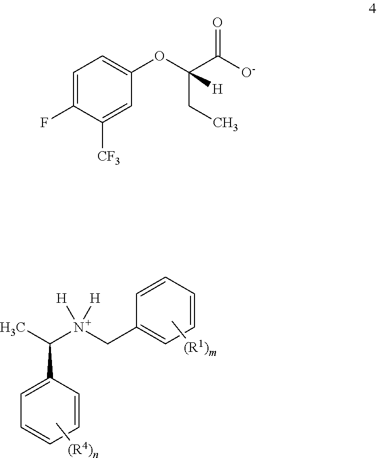

wherein R¹, R², R³, R⁴, m and n are as defined above;

selectively isolating the R,S-salt of Formula 4;

treating the R,S-salt of Formula 4 with a sodium base to provide the carboxylate salt S-5

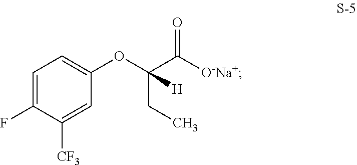

treating carboxylate salt S-5 with acid to prepare compound S-2

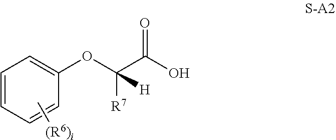

converting compound S-2 to compound S-1.

Embodiment C. This invention also provides a method for preparing a compound of Formula S-A2

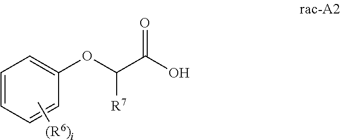

wherein
each R⁶ is independently halogen, nitro, cyano, C₁-C₄ alkyl, C₁-C₄ haloalkyl, C₁-C₄ alkoxy, C₁-C₄ alkenyl, C₁-C₄ haloalkenyl or C₁-C₄ haloalkoxy;

R⁷ is C₁-C₆ alkyl, C₁-C₆ haloalkyl, C₁-C₆ alkenyl or C₁-C₆ haloalkenyl; and

is 0, 1,2 or 3;

the method comprising:

treating the compound of Formula rac-A2

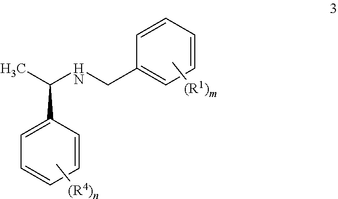

wherein
each R⁶ is independently halogen, nitro, cyano, C₁-C₄ alkyl, C₁-C₄ haloalkyl, C₁-C₄ alkoxy, C₁-C₄ alkenyl, C₁-C₄ haloalkenyl or C₁-C₄ haloalkoxy;

R⁷ is C₁-C₆ alkyl, C₁-C₆ haloalkyl, C₁-C₆ alkenyl or C₁-C₆ haloalkenyl; and

is 0, 1,2 or 3;

with a compound of Formula 3

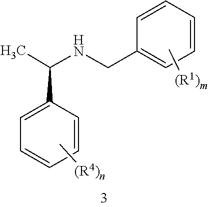

wherein
each R¹ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R²; or
two adjacent R¹ substituents are taken together with the phenyl to which they are attached to form a naphthalenyl ring optionally substituted with up to three R³;
each R² and each R³ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
each R⁴ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R⁵:
each R⁵ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
m is 0, 1, 2 or 3; and
n is 0, 1, 2 or 3;

to provide the R,S-salt of Formula A4

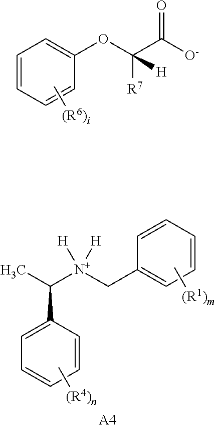

wherein R¹, R², R³, R⁴, R⁵, R⁶, R⁷, i, m and n are as defined above; selectively isolating the R,S-salt of Formula A4;

treating the R,S-salt of Formula A4 with a sodium base to provide a carboxylate salt of Formula S-A5

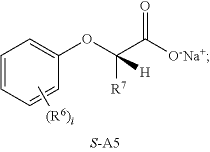

wherein R⁶, R⁷ and i are as defined above; and

treating carboxylate salt of Formula S-A5 with acid.

Embodiment D. This invention also provides a method for racemizing an enantiomerically-enriched compound of Formula scal-A2

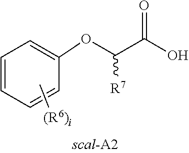

wherein
each R⁶ is independently halogen, nitro, cyano, C₁-C₄ alkyl, C₁-C₄ haloalkyl, C₁-C₄ alkoxy, C₁-C₄ alkenyl, C₁-C₄ haloalkenyl or C₁-C₄ haloalkoxy;
R⁷ is C₁-C₆ alkyl, C₁-C₆ haloalkyl, C₁-C₆ alkenyl or C₁-C₆ haloalkenyl; and

is 0, 1,2 or 3;

the method comprising

treating an enantiomerically-enriched compound of Formula scal-A2 with a chlorinating agent to provide a compound of Formula scal-A12;

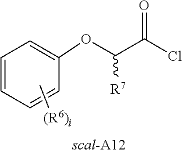

wherein
each R⁶ is independently halogen, nitro, cyano, C₁-C₄ alkyl, C₁-C₄ haloalkyl, C₁-C₄ alkoxy, C₁-C₄ alkenyl, C₁-C₄ haloalkenyl or C₁-C₄ haloalkoxy;
R⁷ is C₁-C₆ alkyl, C₁-C₆ haloalkyl, C₁-C₆ alkenyl or C₁-C₆ haloalkenyl; and

i is 0, 1,2 or 3;

treating the compound of Formula scal-A12 with a tertiary amine to provide a compound of Formula rac-A12

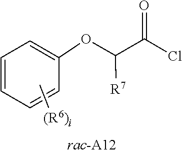

wherein
each R⁶ is independently halogen, nitro, cyano, C₁-C₄ alkyl, C₁-C₄ haloalkyl, C₁-C₄ alkoxy, C₁-C₄ alkenyl, C₁-C₄ haloalkenyl or C₁-C₄ haloalkoxy;
R⁷ is C₁-C₆ alkyl, C₁-C₆ haloalkyl, C₁-C₆ alkenyl or C₁-C₆ haloalkenyl; and
i is 0, 1,2 or 3;

and hydrolyzing the compound of Formula rac-A12 to provide the compound of Formula rac-A2

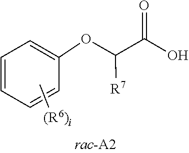

wherein
each R⁶ is independently halogen, nitro, cyano, C₁-C₄ alkyl, C₁-C₄ haloalkyl, C₁-C₄ alkoxy, C₁-C₄ alkenyl, C₁-C₄ haloalkenyl or C₁-C₄ haloalkoxy;
R⁷ is C₁-C₆ alkyl, C₁-C₆ haloalkyl, C₁-C₆ alkenyl or C₁-C₆ haloalkenyl; and

is 0, 1,2 or 3.

Embodiment E. The invention also provides a R,S-salt of Formula A4

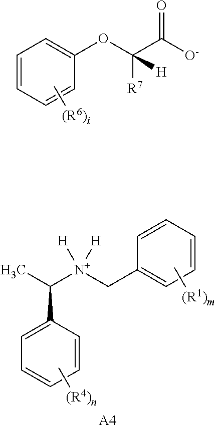

wherein
each R¹ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R²; or
two adjacent R¹ substituents are taken together with the phenyl to which they are attached to form a naphthalenyl ring optionally substituted with up to three R³;
each R² and each R³ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
each R⁴ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R⁵;
each R⁵ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
each R⁶ is independently halogen, nitro, cyano, C₁-C₄ alkyl, C₁-C₄ haloalkyl, C₁-C₄ alkoxy, C₁-C₄ alkenyl, C₁-C₄ haloalkenyl or C₁-C₄ haloalkoxy;
R⁷ is C₁-C₆ alkyl, C₁-C₆ haloalkyl, C₁-C₆ alkenyl or C₁-C₆ haloalkenyl;
i is 0, 1,2 or 3;
m is 0, 1, 2 or 3; and
n is 0, 1,2 or 3.

### DETAILED DESCRIPTION OF THE INVENTION

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition, process or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As used herein, the term “suitable” indicates that the entity or condition so described is appropriate for use in the situation or circumstance indicated. As used herein, the terms “treatment” or treating” denotes using a chemical or chemical process to alter the existing condition of other materials, chemicals or compounds. The terms “converting,” “converted,” “conversion” and the like refer to causing an entity such as a chemical compound to change in structure, form, character or function. For example, a compound of a first formula or structure is converted to a compound of a second formula or structure by a chemical process involving one or more treatments as defined above.

In the above recitations, the term “alkyl”, used either alone or in compound words such as “haloalkyl” and “haloalkenyl” include straight-chain or branched alkyl, such as methyl, ethyl, n-propyl, i-propyl, or the different butyl, pentyl or hexyl isomers. “Alkenyl” includes straight-chain or branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl.

“Alkoxy” includes, for example, methoxy, ethoxy, n-propyloxy, isopropyloxy and the different butoxy, pentoxy and hexyloxy isomers.

The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, or when used in descriptions such as “alkyl substituted with halogen” said alkyl may be partially or fully substituted with halogen atoms which may be the same or different. Examples of “haloalkyl” or “alkyl substituted with halogen” include F₃C, ClCH₂, CF₃CH₂ and CF₃CCl₂. The terms “haloalkoxy”, and the like, is defined analogously to the term “haloalkyl”. Examples of “haloalkoxy” include CF₃O—, CCl₃CH₂O—, HCF₂CH₂CH₂O— and CF₃CH₂O—. “Cyano” denotes a —C≡N group.

The term “tertiary amine” refers to an amine wherein at least one nitrogen atom is substituted with three organic groups such as alkyl groups. Example tertiary amines include 30 triethylamine, di isopropylethylamine, N,N-dimethylbenzylamine, N-methyl morpholine, N-methyl piperidine, N-phenyl piperidine and tetramethylethylene diamine.

As used herein, “alkali metal” refers to elements of group 1 of the periodic table, including lithium, sodium, potassium and cesium, preferably sodium or potassium, or cations thereof, such as when used in combination with an anionic counterion to define a chemical compound.

The total number of carbon atoms in a substituent group is indicated by the “Cᵢ-Cⱼ” prefix where i and j are numbers from 1 to 6. When a compound is substituted with a substituent bearing a subscript that indicates the number of said substituents can exceed 1, said substituents (when they exceed 1) are independently selected from the group of defined substituents, (e.g., (R¹)ₘ, m is 0, 1, 2 or 3). When a group contains a substituent that can be hydrogen, for example (when m=0), then when this substituent is taken as hydrogen, it is recognized that this is equivalent to said group being unsubstituted. When a variable group is shown to be optionally attached to a position, (for example (R¹)ₘ attached to a phenyl wherein m may be 0, then hydrogen may be at the position even if not recited in the variable group definition. When one or more positions on a group are said to be “not substituted” or “unsubstituted”, then hydrogen atoms are attached to take up any free valency.

As used herein, “adjacent” means that two substituents are near each other but are not directly connected. For example, the term “adjacent R¹ substituents” indicates R¹ substituents that are attached to contiguous carbon atoms, such as in a phenyl group.

The term “optionally” when used herein means that the optional condition may or may not be present. For example, when a reaction is conducted optionally in the presence of a solvent, the solvent may or may not be present.

The term “optionally substituted” refers to groups which are unsubstituted or have at least one non-hydrogen substituent that does not extinguish the chemical or biological activity possessed by the unsubstituted analog. As used herein, the following definitions shall apply unless otherwise indicated. The term “optionally substituted with” is used interchangeably with the phrase “unsubstituted or substituted with” or with the term “(un)substituted with”.

Unless otherwise indicated, an optionally substituted group may have a substituent at each substitutable position of the group, and each substitution is independent of the other.

As used herein, the term “selectively isolating” refers to separating a desired compound from other compound(s) in a mixture. “Selectively isolating” includes using chromatography, distillation, extraction and/or crystallization. In particular, “selectively isolating” refers to separating the R,S-salts described herein from R,R-salts. Preferably, the desired R,S-salts described herein may be selectively isolated from the R,R-salts by crystallization.

This invention comprises racemic mixtures, for example, essentially equal amounts of the enantiomers of 2-bromobutanoic acid. In addition, this invention includes compounds that are enantiomerically enriched compared to the racemic mixture; for example in an enantiomer of the compound of Formula S-1 or any intermediate in a process described herein for preparing the compound of Formula S-1. Also included are the essentially pure enantiomers of compounds of Formula S-1 or any intermediate in a process described herein for preparing the compound of Formula S-1.

When enantiomerically enriched, one enantiomer is present in greater amounts than the other, and the extent of enrichment can be defined by an expression of enantiomeric excess (“ee”), which is defined as (Fₘₐⱼ−Fₘᵢₙ)·100%, where Fₘₐⱼ is the mole fraction of the dominant enantiomer in the mixture and Fₘᵢₙ is the mole fraction of the lesser enantiomer in the mixture (e.g., an ee of 20% corresponds to a 60:40 ratio of enantiomers).

As used herein, compounds having at least an 80% enantiomeric excess; preferably at least a 90% enantiomeric excess; more preferably at least a 94% enantiomeric excess, at least a 96% enantiomeric excess; at least a 98% enantiomeric excess of a specific isomer are designated as R— or S—, depending on the predominant configuration at the asymmetric center. Of note are essentially enantiomerically pure embodiments (>99% ee) of the more predominant enantiomer. As used herein, compounds having less than 80% enantiomeric excess are designated as scalemic.

Molecular depictions drawn herein generally follow standard conventions for depicting stereochemistry. To indicate stereoconfiguration, bonds rising from the plane of the drawing and towards the viewer are denoted by solid wedges where the broad end of the wedge is attached to the atom rising from the plane of the drawing towards the viewer as shown below, where group B is rising from above the plane of the drawing. Except where specifically indicated, hydrogen atoms attached to the asymmetric center are generally not shown.

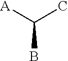

Bonds going below the plane of the drawing and away from the viewer are denoted by dashed wedges where the broad end of the wedge is attached to the atom further away from the viewer, i.e. group B′ is below the plane of the drawing.

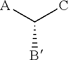

Constant width lines indicate bonds with a direction opposite or neutral relative to bonds shown with solid or dashed wedges; constant width lines also depict bonds in molecules or parts of molecules in which no stereoconfiguration is intended to be specified. Notably as used herein, a constant width line attached to an asymmetric center also represents a condition where the amounts of R— and S-configuration at that center are essentially equal; e.g., a compound with a single asymmetric center is racemic. When a racemic mixture is intended for any specific compound herein, it is denoted with the prefix “rac-”

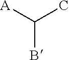

Racemic mixture or “rac”

Wavy lines indicate bonds in molecules or parts of molecules in which no particular stereoconfiguration is intended to be specified. Accordingly, as used herein, a wavy line attached to an asymmetric center represents a condition where the amounts of R— and S—configuration at that center are non-equal but not of sufficiently high enantiomeric excess for either R— or S-configuration; e.g., a compound with a single asymmetric center is scalemic as defined herein. When a scalemic mixture is intended for any specific compound herein, it is denoted with the prefix “scal-”

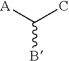

Scalemic mixture or “scal-”

Embodiments of the invention include the following.

Embodiment A1. The method of Embodiment A wherein m is 0, 1 or 2.

Embodiment A2. The method of Embodiment A1 wherein m is 1 or 2.

Embodiment A3. The method of Embodiment A, Embodiment A1 or Embodiment A2 wherein

each R¹ is independently halogen, nitro, C₁-C₄ alkyl, C₁-C₄ haloalkyl; or phenyl optionally substituted with one R²; or

two adjacent R¹ substituents are taken together with the phenyl to which they are attached to form a naphthalenyl ring optionally substituted with up to two R³.

Embodiment A4. The method of any of Embodiments A through A3 wherein each R¹ is independently halogen, nitro, C₁-C₄ alkyl or C₁-C₄ haloalkyl.

Embodiment A5. The method of Embodiment A4 wherein each R¹ is independently halogen or C₁-C₄ alkyl.

Embodiment A6. The method of Embodiment A5 wherein m is 2 and the R¹ substituents are chloro at the 2—and 4-positions.

Embodiment A7. The method of any of Embodiments A through A6 wherein n is 0, 1 or 2.

Embodiment A8. The method of Embodiment A7 wherein n is 0.

Embodiment A9. The method of Embodiment A7 wherein n is 1 or 2.

Embodiment A10. The method of Embodiment A9 wherein each R⁴ is independently halogen, nitro, C₁-C₄ alkyl or C₁-C₄ haloalkyl.

Embodiment A11. The method of Embodiment A10 wherein each R⁴ is independently halogen, or C₁-C₄ alkyl.

Embodiment A12. The method of any of Embodiments A through A11 wherein the compound of Formula 3 is selected from the group consisting of

(αR)-α-methyl-N-(phenylmethyl)-benzenemethanamine,

N-[(1R)-1-phenylethyl]-1-naphthalenemethanamine,

2,4-dichloro-N-[(1R)-1-phenylethyl]-benzenemethanamine,

3,4-dichloro-N-[(1R)-1-phenylethyl]-benzenemethanamine,

2,6-dichloro-N-[(1R)-1-phenylethyl]-benzenemethanamine,

2,4,6-trimethyl-N-[(1R)-1-phenylethyl]-benzenemethanamine,

4-nitro-N-[(1R)-1-phenylethyl]-benzenemethanamine, and

2-methyl-3-phenyl-N-[(1R)-1-phenylethyl]-benzenemethanamine.

Embodiment A13. The method of Embodiment A12 wherein m is 2 and the R¹ substituents are chloro at the 2—and 4-positions; and n is 0; i.e. the compound of Formula 3 is compound 3A [2,4-dichloro-N-[(1R)-1-phenylethyl]-benzenemethanamine, CAS No. 1409308-40-2]

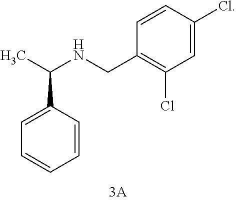 [SMILES: C[C@@H](NCc1ccc(Cl)cc1Cl)c1ccccc1]

Embodiment A14. The method of any of Embodiments A through A13 further comprising wherein compound S-1 is prepared from compound S-2 by the method comprising

treating compound S-2 with a chlorinating agent to prepare compound S-6

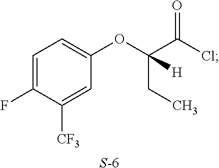 [SMILES: CC[C@H](Oc1ccc(F)c(C(F)(F)F)c1)C(=O)Cl ; "S-6"]

and treating compound S-6 with compound 7 (i.e. benzylamine)

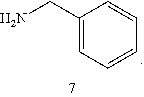

Embodiment A15. The method of Embodiment A14 wherein the chlorinating agent is thionyl chloride.

Embodiment A16. The method of any of Embodiments A through A15 wherein compound rac-2 is prepared by the method comprising treating a compound of Formula rac-8

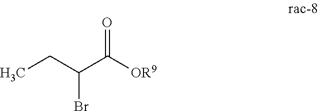

wherein R⁹ is C₁-C₆ alkyl;
with compound 9 (i.e. 4-fluoro-3-(trifluoromethyl)phenol) in the presence of a base

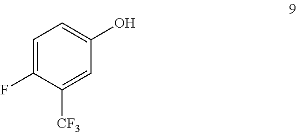 [SMILES: Oc1ccc(F)c(C(F)(F)F)c1]

to provide a compound of Formula rac-10

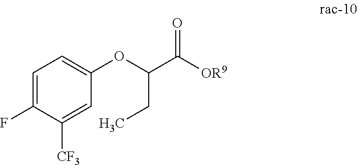

wherein R⁹ is C₁-C₆ alkyl;

and hydrolyzing a compound of Formula rac-10.

Embodiment A17. The method of Embodiment A16 wherein R⁹ is CH₃.

Embodiment B1. The method of Embodiment B wherein m is 0, 1 or 2.

Embodiment B2. The method of Embodiment B1 wherein m is 1 or 2.

Embodiment B3. The method of Embodiment B, Embodiment B1 or Embodiment B2 wherein

each R¹ is independently halogen, nitro, C₁-C₄ alkyl, C₁-C₄ haloalkyl; or phenyl optionally substituted with one R²; or

two adjacent R¹ substituents are taken together with the phenyl to which they are attached to form a naphthalenyl ring optionally substituted with up to two R³.

Embodiment B4. The method of any of Embodiments B through B3 wherein each R¹ is independently halogen, nitro, C₁-C₄ alkyl or C₁-C₄ haloalkyl.

Embodiment B5. The method of Embodiment B4 wherein each R¹ is independently halogen or C₁-C₄ alkyl.

Embodiment B6. The method of Embodiment B5 wherein m is 2 and the R¹ substituents are chloro in the 2—and 4-positions.

Embodiment B7. The method of any of Embodiments B through B6 wherein n is 0, 1 or 2.

Embodiment B8. The method of Embodiment B7 wherein n is 0.

Embodiment B9. The method of Embodiment B7 wherein n is 1 or 2.

Embodiment B10. The method of Embodiment B9 wherein each R⁴ is independently halogen, nitro, C₁-C₄ alkyl or C₁-C₄ haloalkyl.

Embodiment B11. The method of Embodiment B10 wherein each R⁴ is independently halogen, or C₁-C₄ alkyl.

Embodiment B12. The method of any of Embodiments B through B11 wherein the compound of Formula 3 is selected from the group consisting of

(αR)-α-methyl-N-(phenylmethyl)-benzenemethanamine,

N-[(1R)-1-phenylethyl]-1-naphthalenemethanamine,

2,4-dichloro-N-[(1R)-1-phenylethyl]-benzenemethanamine,

3,4-dichloro-N-[(1R)-1-phenylethyl]-benzenemethanamine,

2,6-dichloro-N-[(1R)-1-phenylethyl]-benzenemethanamine,

2,4,6-trimethyl-N-[(1R)-1-phenylethyl]-benzenemethanamine,

4-nitro-N-[(1R)-1-phenylethyl]-benzenemethanamine, and

2-methyl-3-phenyl-N-[(1R)-1-phenylethyl]-benzenemethanamine.

Embodiment B13. The method of Embodiment B12 wherein m is 2 and the R¹ substituents are chloro in the 2— and 4-positions; and n is 0; i.e. the compound of Formula 3 is compound 3A [2,4-dichloro-N-[(1R)-1-phenylethyl]-benzenemethanamine, CAS No. 1409308-40-2]

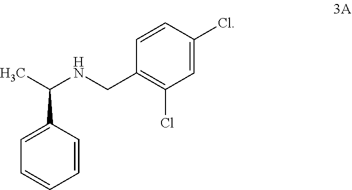 [SMILES: C[C@@H](NCc1ccc(Cl)cc1Cl)c1ccccc1]

Embodiment B14. The method of any of Embodiments B through B13 wherein converting compound S-2 to compound S-1 comprises

treating compound S-2 with a chlorinating agent to prepare compound S-6

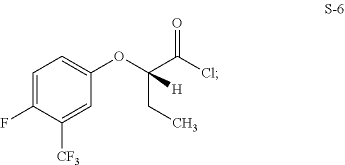 [SMILES: CC[C@H](Oc1ccc(F)c(C(F)(F)F)c1)C(=O)Cl]

and treating compound S-6 with compound 7

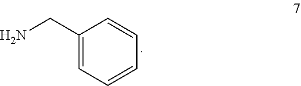

Embodiment B15. The method of Embodiment B14 wherein the chlorinating agent is thionyl chloride.

Embodiment B16. The method of any of Embodiments B through B15 wherein compound rac-2 is prepared by treating a compound of Formula rac-8

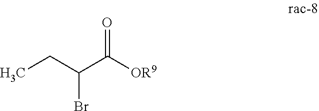

wherein R⁹ is C₁-C₆ alkyl;

with compound 9 in the presence of a base

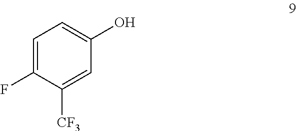 [SMILES: Oc1ccc(F)c(C(F)(F)F)c1]

to provide a compound of Formula_rac-10

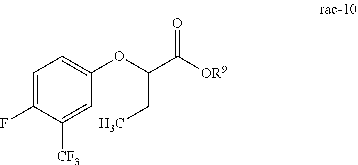

wherein R⁹ is C₁-C₆ alkyl; and hydrolyzing the compound of Formula rac-10.

Embodiment B17. The method of Embodiment B16 wherein R⁹ is CH₃.

Embodiment C1. The method of Embodiment C wherein m is 0, 1 or 2.

Embodiment C2. The method of Embodiment C₁ wherein m is 1 or 2.

Embodiment C3. The method of Embodiment C, Embodiment C1 or Embodiment C2 wherein

each R¹ is independently halogen, nitro, C₁-C₄ alkyl, C₁-C₄ haloalkyl; or phenyl optionally substituted with one R²; or

two adjacent R¹ substituents are taken together with the phenyl to which they are attached to form a naphthalenyl ring optionally substituted with up to two R³.

Embodiment C4. The method of any of Embodiments C through C3 wherein each R¹ is independently halogen, nitro, C₁-C₄ alkyl or C₁-C₄ haloalkyl.

Embodiment C5. The method of Embodiment C4 wherein each R¹ is independently halogen or C₁-C₄ alkyl.

Embodiment C₆. The method of Embodiment C5 wherein m is 2 and the R¹ substituents are chloro at the 2— and 4-positions.

Embodiment C7. The method of any of Embodiments C through C₆ wherein n is 0, 1 or 2.

Embodiment C8. The method of Embodiment C7 wherein n is 0.

Embodiment C9. The method of Embodiment C7 wherein n is 1 or 2.

Embodiment C1O. The method of Embodiment C9 wherein each R⁴ is independently halogen, nitro, C₁-C₄ alkyl or C₁-C₄ haloalkyl.

Embodiment C11. The method of Embodiment C10 wherein each R⁴ is independently halogen, or C₁-C₄ alkyl.

Embodiment C12. The method of any of Embodiments C through C11 wherein the compound of Formula 3 is selected from the group consisting of

(αR)-α-methyl-N-(phenylmethyl)-benzenemethanamine,

N-[(1R)-1-phenylethyl]-1-naphthalenemethanamine,

2,4-dichloro-N-[(1R)-1-phenylethyl]-benzenemethanamine,

3,4-dichloro-N-[(1R)-1-phenylethyl]-benzenemethanamine,

2,6-dichloro-N-[(1R)-1-phenylethyl]-benzenemethanamine,

2,4,6-trimethyl-N-[(1R)-1-phenylethyl]-benzenemethanamine,

4-nitro-N-[(1R)-1-phenylethyl]-benzenemethanamine, and

2-methyl-3-phenyl-N-[(1R)-1-phenylethyl]-benzenemethanamine.

Embodiment C13. The method of Embodiment C12 wherein m is 2 and the R¹ substituents are chloro in the 2— and 4-positions, and n is 0; i.e. the compound of Formula 3 is compound 3A [2,4-dichloro-N-[(1R)-1-phenylethyl]-benzenemethanamine, CAS No. 1409308-40-2]

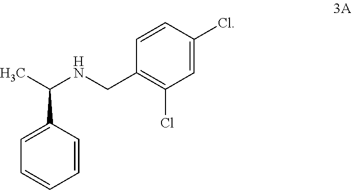 [SMILES: C[C@@H](NCc1ccc(Cl)cc1Cl)c1ccccc1]

Embodiment C14. The method of any of Embodiments C through C13 wherein i is 0, 1 or 2.

Embodiment C15. The method of Embodiment C14 wherein i is 0.

Embodiment C16. The method of Embodiment C14 wherein i is 1 or 2.

Embodiment C17. The method of Embodiment C16 wherein each R⁶ is independently halogen, C₁-C₄ alkyl, C₁-C₄ alkoxy or C₁-C₄ haloalkyl.

Embodiment C18. The method of Embodiment C17 wherein each R⁶ is independently halogen, C₁-C₄ alkyl or C₁-C₄ haloalkyl.

Embodiment C19. The method of Embodiment C18 wherein each R⁶ is independently halogen or C₁-C₄ haloalkyl.

Embodiment C20. The method any of Embodiments C through C21 wherein R⁷ is C₁-C₆ alkyl.

Embodiment C21. The method Embodiment C20 wherein R⁷ is C₁-C₂ alkyl.

Embodiment C22. The method of any of Embodiments C through C21 wherein i is 2, one R⁶ is 3-CF₃, the second R⁶ is 4-F and R⁷ is ethyl; i.e. the compound of Formula S-A2 is compound S-2

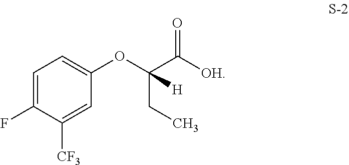 [SMILES: CC[C@H](Oc1ccc(F)c(C(F)(F)F)c1)C(=O)O]

Embodiment D1. The method of Embodiment D wherein i is 0, 1 or 2.

Embodiment D2. The method of Embodiment D1 wherein i is 0.

Embodiment D3. The method of Embodiment D2 wherein i is 1 or 2.

Embodiment D4. The method of Embodiment D3 wherein each R⁶ is independently halogen, C₁-C₄ alkyl, C₁-C₄ alkoxy or C₁-C₄ haloalkyl.

Embodiment D5. The method of Embodiment D4 wherein each R⁶ is independently halogen, C₁-C₄ alkyl or C₁-C₄ haloalkyl.

Embodiment D6. The method of Embodiment D5 wherein each R⁶ is independently halogen or C₁-C₄ haloalkyl.

Embodiment D7. The method any of Embodiments D through D6 wherein R⁷ is C₁-C₆ alkyl.

Embodiment D8. The method Embodiment D7 wherein R⁷ is C₁-C₂ alkyl.

Embodiment D9. The method of any of Embodiments D through D8 wherein i is 2, one R⁶ is 3-CF₃, the second R⁶ is 4-F and R⁷ is ethyl; i.e. the compound of Formula scal-A2 is Formula scal-A2A

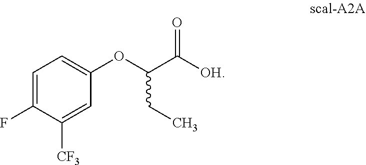 [SMILES: CCC(Oc1ccc(F)c(C(F)(F)F)c1)C(=O)O]

Embodiment D10. The method of any of Embodiments D through D9 wherein the tertiary amine is triethylamine.

Embodiment D11. The method of any of Embodiments D through D10 wherein the chlorinating agent is thionyl chloride.

Embodiment D12. The method of any of Embodiments D through D11 wherein the compound of Formula scal-A2 is predominantly the R-enantiomer.

Embodiment E1. The R,S-salt of Embodiment E wherein m is 0, 1 or 2.

Embodiment E2. The R,S-salt of Embodiment E1 wherein m is 1 or 2.

Embodiment E3. The R,S-salt of Embodiment E, Embodiment E1 or Embodiment E2 wherein

each R¹ is independently halogen, nitro, C₁-C₄ alkyl, C₁-C₄ haloalkyl; or phenyl optionally substituted with one R²; or

two adjacent R¹ substituents are taken together with the phenyl to which they are attached to form a naphthalenyl ring optionally substituted with up to two R³.

Embodiment E4. The R,S-salt of any of Embodiments E through E3 wherein each R¹ is independently halogen, nitro, C₁-C₄ alkyl or C₁-C₄ haloalkyl.

Embodiment E5. The R,S-salt of Embodiment E4 wherein each R¹ is independently halogen or C₁-C₄ alkyl.

Embodiment E6. The R,S-salt of Embodiment E5 wherein m is 2 and the R¹ substituents are chloro at the 2— and 4-positions.

Embodiment E7. The R,S-salt of any of Embodiments E through E6 wherein n is 0, 1 or 2.

Embodiment E8. The R,S-salt of Embodiment E7 wherein n is 0.

Embodiment E9. The R,S-salt of Embodiment E7 wherein n is 1 or 2.

Embodiment E10. The R,S-salt of Embodiment E9 wherein each R⁴ is independently halogen, nitro, C₁-C₄ alkyl or C₁-C₄ haloalkyl.

Embodiment E11. The R,S-salt of Embodiment E10 wherein each R⁴ is independently halogen, or C₁-C₄ alkyl.

Embodiment E12. The R,S-salt of any of Embodiments E through E11 wherein the compound of Formula 3 is selected from the group consisting of

(αR)-α-methyl-N-(phenylmethyl)-benzenemethanamine,

N-[(1R)-1-phenylethyl]-1-naphthalenemethanamine,

2,4-dichloro-N-[(1R)-1-phenylethyl]-benzenemethanamine,

3,4-dichloro-N-[(1R)-1-phenylethyl]-benzenemethanamine,

2,6-dichloro-N-[(1R)-1-phenylethyl]-benzenemethanamine,

2,4,6-trimethyl-N-[(1R)-1-phenylethyl]-benzenemethanamine,

4-nitro-N-[(1R)-1-phenylethyl]-benzenemethanamine, and

2-methyl-3-phenyl-N-[(1R)-1-phenylethyl]-benzenemethanamine.

Embodiment E13. The R,S-salt of Embodiment E12 wherein m is 2 and the R¹ substituents are chloro in the 2— and 4-positions, and n is 0; i.e. the R,S-salt comprises compound 3A [2,4-dichloro-N-[(1R)-1-phenylethyl]-benzenemethanamine, CAS No. 1409308-40-2]

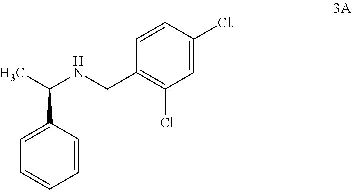 [SMILES: C[C@@H](NCc1ccc(Cl)cc1Cl)c1ccccc1]

Embodiment E14. The R,S-salt of any of Embodiments E through E13 wherein i is 0, 1 or 2.

Embodiment E15. The R,S-salt of Embodiment E14 wherein i is 0.

Embodiment E16. The R,S-salt of Embodiment E14 wherein i is 1 or 2.

Embodiment E17. The R,S-salt of Embodiment E16 wherein each R⁶ is independently halogen, C₁-C₄ alkyl, C₁-C₄ alkoxy or C₁-C₄ haloalkyl.

Embodiment E18. The R,S-salt of Embodiment E17 wherein each R⁶ is independently halogen, C₁-C₄ alkyl or C₁-C₄ haloalkyl.

Embodiment E19. The R,S-salt of Embodiment E18 wherein each R⁶ is independently halogen or C₁-C₄ haloalkyl.

Embodiment E20. The method any of Embodiments E through E19 wherein R⁷ is C₁-C₆ alkyl.

Embodiment E21. The method Embodiment E20 wherein R⁷ is C₁-C₂ alkyl.

Embodiment E22. The method of any of Embodiments E through E13 wherein i is 2; m is 2; n is 0; the R¹ substituents are chloro at the 2— and 4-positions; one R⁶ is 3-CF₃, the second R⁶ is 4-F and R⁷ is ethyl; i.e. the compound of Formula A4 is the R,S-salt of Formula 4B

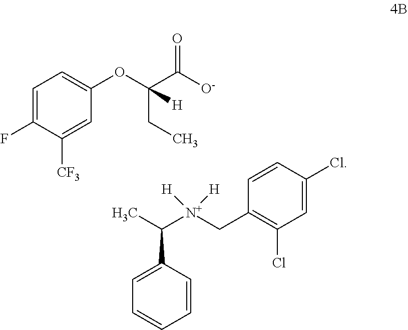 [SMILES: CC[C@H](Oc1ccc(F)c(C(F)(F)F)c1)C(=O)[O-] ; C[C@@H]([NH2+]Cc1ccc(Cl)cc1Cl)c1ccccc1]

Embodiments of this invention, including Embodiments A1 through A17, B1 through B17, C₁ through C22, D1 through D12 and E1 through E22 above as well as any other embodiments (including Embodiments 1 through 19) described herein, can be combined in any manner, and the descriptions of variables in the embodiments pertain not only to the compounds of Formula S-1 but also to the starting compounds and intermediate compounds of Formulae 2 through 11, useful for preparing compound S-1.

### Preferred Embodiments Include the Following

Embodiment 1. The method of any of Embodiments A, B or C wherein

m is 1 or 2;

n is 0; and

each R¹ is independently halogen, nitro, C₁-C₄ alkyl, C₁-C₄ haloalkyl; or phenyl optionally substituted with one R²; or

two adjacent R¹ substituents are taken together with the phenyl to which they are attached to form a naphthalenyl ring optionally substituted with up to two R³.

Embodiment 2. The method of any of Embodiments A, B or C wherein each R¹ is independently halogen, nitro, C₁-C₄ alkyl or C₁-C₄ haloalkyl.

Embodiment 3. The method of any of Embodiments A, B or C wherein the compound of Formula 3 is selected from the group consisting of

(αR)-α-methyl-N-(phenylmethyl)-benzenemethanamine,

N-[(1R)-1-phenylethyl]-1-naphthalenemethanamine,

2,4-dichloro-N-[(1R)-1-phenylethyl]-benzenemethanamine,

3,4-dichloro-N-[(1R)-1-phenylethyl]-benzenemethanamine,

2,6-dichloro-N-[(1R)-1-phenylethyl]-benzenemethanamine,

2,4,6-trimethyl-N-[(1R)-1-phenylethyl]-benzenemethanamine,

4-nitro-N-[(1R)-1-phenylethyl]-benzenemethanamine, and

2-methyl-3-phenyl-N-[(1R)-1-phenylethyl]-benzenemethanamine.

Embodiment 4. The method of Embodiments A or B for preparing the compound of Formula S-1 from the compound of Formula S-2 further comprising treating compound S-2 with a chlorinating agent to prepare compound S-6

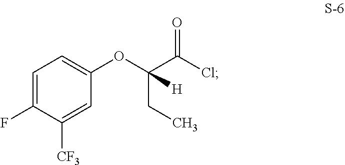 [SMILES: CC[C@H](Oc1ccc(F)c(C(F)(F)F)c1)C(=O)Cl]

and treating compound S-6 with a compound of Formula 7

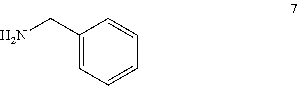

to prepare compound S-1.

Embodiment 5. The method of Embodiments A or B wherein compound rac-2 is prepared by treating a compound of Formula rac-8

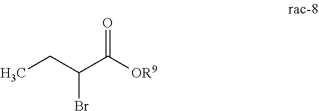

wherein R⁹ is C₁-C₆ alkyl;

with compound 9 in the presence of a base

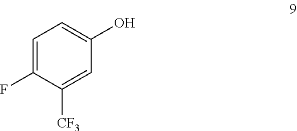 [SMILES: Oc1ccc(F)c(C(F)(F)F)c1]

to provide a compound of Formula rac-10

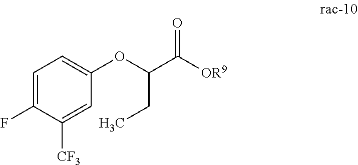

wherein R⁹ is C₁-C₆ alkyl; and

hydrolyzing the compound of Formula rac-10.

Embodiment 6. The method of Embodiments A or B wherein converting compound S-2 to compound S-1 comprises

treating compound S-2 with a chlorinating agent to prepare compound S-6

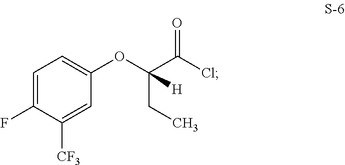 [SMILES: CC[C@H](Oc1ccc(F)c(C(F)(F)F)c1)C(=O)Cl]

and treating compound S-6 with compound 7

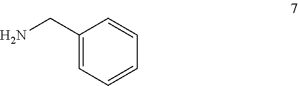

to prepare compound S-1.

Embodiment 7. The method of Embodiment C for preparing a compound of Formula S-A2 wherein

each R⁶ is independently halogen, C₁-C₄ alkyl, C₁-C₄ alkoxy or C₁-C₄ haloalkyl;

R⁷ is C₁-C₆ alkyl; and

i is 0, 1 or 2.

Embodiment 8. The method of Embodiment 7 wherein

each R⁶ is independently halogen, C₁-C₄ alkyl or C₁-C₄ haloalkyl; and

R⁷ is C₁-C₂ alkyl.

Embodiment 9. The method of Embodiment 8 wherein i is 2; one R⁶ is 3-CF₃, the second R⁶ is 4-F; and R⁷ is ethyl.

Embodiment 10. The method of Embodiment D for racemizing an enantiomerically-enriched compound of Formula scal-A2 wherein

each R⁶ is independently halogen, C₁-C₄ alkyl, C₁-C₄ alkoxy or C₁-C₄ haloalkyl;

R⁷ is C₁-C₆ alkyl; and

i is 0, 1 or 2.

Embodiment 11. The method of Embodiment 10 wherein

each R⁶ is independently halogen, C₁-C₄ alkyl or C₁-C₄ haloalkyl; and

R⁷ is C₁-C₂ alkyl.

Embodiment 12. The method of Embodiment 11 wherein i is 2; one R⁶ is 3-CF₃, the second R⁶ is 4-F; and R⁷ is ethyl.

Embodiment 13. The R,S-salt of Formula A4 wherein

m is 1 or 2;

n is 0; and

each R¹ is independently halogen, nitro, C₁-C₄ alkyl, C₁-C₄ haloalkyl; or phenyl; or

two adjacent R¹ substituents are taken together with the phenyl to which they are attached to form an unsubstituted naphthalenyl ring.

Embodiment 14. The R,S-salt of Formula A4 wherein each R¹ is independently halogen, nitro, C₁-C₄ alkyl or C₁-C₄ haloalkyl.

Embodiment 15. The R,S-salt of Formula A4 that is a salt of an amine selected from the group consisting of

(αR)-α-methyl-N-(phenylmethyl)-benzenemethanamine,

N-[(1R)-1-phenylethyl]-1-naphthalenemethanamine,

2,4-dichloro-N-[(1R)-1-phenylethyl]-benzenemethanamine,

3,4-dichloro-N-[(1R)-1-phenylethyl]-benzenemethanamine,

2,6-dichloro-N-[(1R)-1-phenylethyl]-benzenemethanamine,

2,4,6-trimethyl-N-[(1R)-1-phenylethyl]-benzenemethanamine,

4-nitro-N-[(1R)-1-phenylethyl]-benzenemethanamine, and

2-methyl-3-phenyl-N-[(1R)-1-phenylethyl]-benzenemethanamine.

Embodiment 16. The R,S-salt of Formula A4 wherein m is 2 and the R¹ substituents are chloro at the 2— and 4-positions; and n is 0.

Embodiment 17. The R,S-salt of Formula A4 wherein

each R⁶ is independently halogen, C₁-C₄ alkyl, C₁-C₄ alkoxy or C₁-C₄ haloalkyl;

and

R⁷ is C₁-C₆ alkyl.

Embodiment 18. The R,S-salt of Formula A4 wherein

each R⁶ is independently halogen, C₁-C₄ alkyl or C₁-C₄ haloalkyl; and

R⁷ is C₁-C₂ alkyl.

Embodiment 19. The R,S-salt of Formula A4 wherein i is 2; one R⁶ is 3-CF₃, the second R⁶ is 4-F and R⁷ is ethyl.

Embodiment 20. The R,S-salt of Formula A4 wherein

m is 2 and the R¹ substituents are chloro in the 2— and 4-positions;

n is 0;

i is 2, one R⁶ is 3-CF₃ and the second R⁶ is 4-F; and

R⁷ is ethyl; i.e. the R,S salt 4B

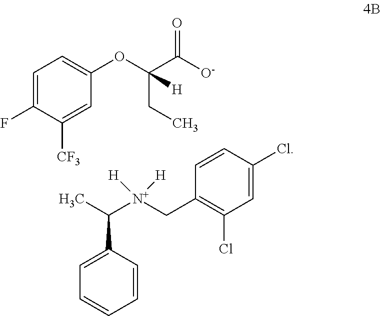 [SMILES: CC[C@H](Oc1ccc(F)c(C(F)(F)F)c1)C(=O)[O-] ; C[C@@H]([NH2+]Cc1ccc(Cl)cc1Cl)c1ccccc1]

In the following Schemes the definitions of R¹, R², R³, R⁴, R⁵, R⁶, R⁷, R⁸, R⁹, i, m and n in the compounds of Formulae 3 through 11 below are as defined above in the Summary of the Invention and description of embodiments unless otherwise indicated.

The methods described herein provide an efficient and robust synthesis of compound S-1.

As summarized in Scheme 1, compound S-1 can be prepared from compound S-2, wherein compound S-2 is obtained by resolution of compound rac-2, as described in greater detail with reference to Scheme 2. Conversion of compound S-2 to compound S-1 can be accomplished by any of several reaction sequences subsequently described herein.

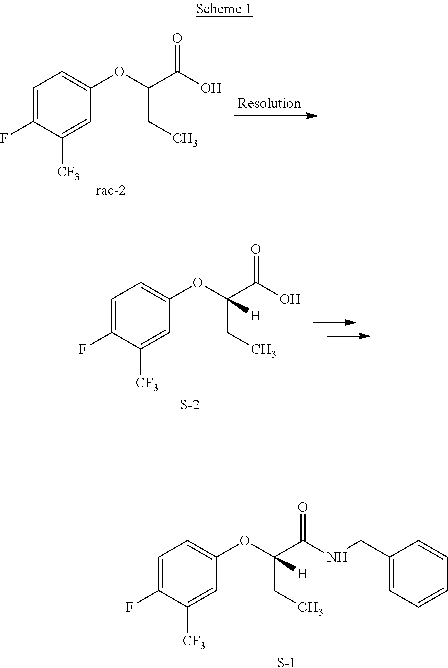

Obtaining acids of high enantiomeric purity can be accomplished in several ways, including catalytic asymmetric synthesis, chromatographic resolution, extraction resolution, membrane resolution, enzymatic resolution and diastereomeric salt resolution. Optical resolution of racemic substrates through diastereomeric salt formation is one of the more practical and economical approaches for industrial-scale production. However, the efficiency of diasteromeric salt resolutions depends on the differential solubility of the diasteromeric salts in at least one solvent. For a given racemate, finding a suitable resolving agent/solvent combination is largely a matter of trial and error, a time-consuming and labor-intensive process. Obtaining a high enantiomeric excess may also require multiple recrystallizations of the diastereomeric salt, which can be very detrimental to industrial processes.

U.S. Pat. No. 4,929,273 describes the resolution of the compound of Formula rac-2 using L-(−)-α-phenylethylmine. The resolution required repeated recrystallizations of the diastereomeric salt from carbon tetrachloride to obtain the compound of Formula S-2 with high ee. Resolution of 4-chloromandelic acid using R-(+)-benzyl-1-phenylethylamine has been disclosed (Molecules 2018, 23, 3354).

As shown in Scheme 2, resolution of compound rac-2 can be achieved with high efficiency by treatment with a compound of Formula 3, having the R-configuration at the asymmetric center. Treatment of compound rac-2 with a compound of Formula 3 provides the R,R— and R,S-diastereomeric salts of the compound of Formula 3 with either the R— or S—configuration of the acid of compound rac-2, respectively. For compound rac-2, equal amounts of the R,R— and R,S-diastereomeric salts are formed. The desired R,S-salt, the compound of Formula 4, is selectively isolated from the R,R-salt (not shown), preferably by crystallization from a solvent or combination of solvents. Suitable solvents include ketones such as acetone and methyl isobutyl ketone (MIBK), alcohols, optionally mixed with water, such as methanol, ethanol and isopropanol, polar aprotic solvents such as acetonitrile and ethyl acetate, and hydrocarbons such as hexane, petroleum ether, heptane and toluene, and mixtures thereof. The R,S-diastereomeric salt of Formula 4 is generally the less soluble or more stable salt and can be selectively isolated by filtration. Desirably, the isolation provides the R,S salt compound of Formula 4 in high enantiomeric purity and high yield in a single crystallization.

The resulting solid R,S salt of Formula 4 is treated with aqueous base, such as sodium hydroxide, to provide the water-soluble sodium salt compound S-5. Extraction with organic solvents such as toluene can recover the resolving agent (i.e. a compound of Formula 3) for use in subsequent resolutions. Treatment of the compound of Formula R-5 (not shown) with an acid such as hydrochloric acid provides the compound of Formula R-2 (not shown), which can be extracted from the aqueous phase with a suitable organic solvent, such as toluene.

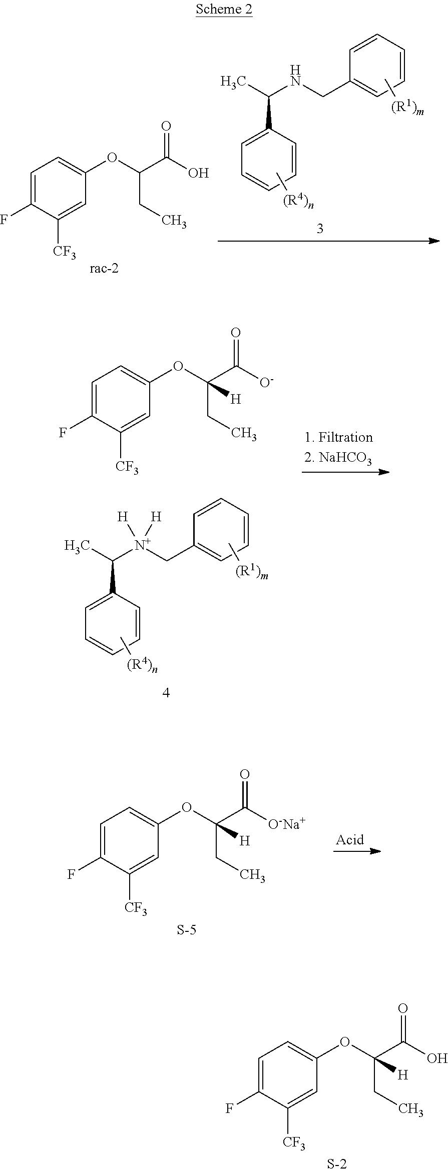

As shown in Scheme 3, a compound of Formula 3 can be prepared by treatment of optionally substituted (R)-1-phenylethylamine with the desired optionally substituted benzyl halide or naphthalenylmethyl halide, typically in the presence of an additional base such as potassium carbonate, and optionally in a suitable solvent. Certain compounds of Formula 3 are disclosed in JP2005023055. Suitable additional bases for the reaction include alkali metal alkoxides such as sodium isopropoxide and potassium tert-butoxide; or alkali metal hydroxides such as potassium hydroxide and sodium hydroxide; or alkali metal carbonates and bicarbonates such as sodium bicarbonate, potassium bicarbonate, sodium carbonate, potassium carbonate and cesium carbonate. A preferred base is potassium carbonate. Suitable solvents include acetonitrile, dichloromethane, dichloroethane, toluene, tetrahydrofuran, dimethyl sulfoxide or N,N-dimethylformamide. Preferred solvents include N,N dimethylformamide.

Preferred compounds of Formula 3 include those wherein n is 0 and/or each R¹ is independently halogen, nitro, C₁-C₄ alkyl or phenyl; or two R¹ substituents are taken together with the phenyl to which they are attached to form an unsubstituted naphthalenyl ring. Of note is the compound of Formula 3 wherein m is 2; n is 0; and the R¹ substituents are chloro at the 2— and 4-positions of the phenyl, (i.e. compound 3A) preferably when used with heptane as a solvent. Using the most preferred combination of compound 3A with heptane, compound S-2 can be obtained, after workup described below, in 45% yield (90% of the available R— enantiomer in rac-2) with 96% ee without the need for recrystallization of the salt of Formula 4.

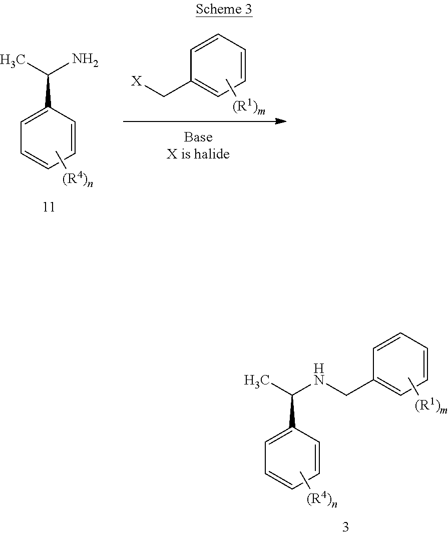

The resolution summarized in Scheme 2 can be generalized for resolving other compounds of Formula rac-A2 as shown in Scheme 4. The selection of the compound of Formula 3 and solvent system used for a specific resolution is dependent on the specific substituents on the racemic substrate of Formula rac-A2.

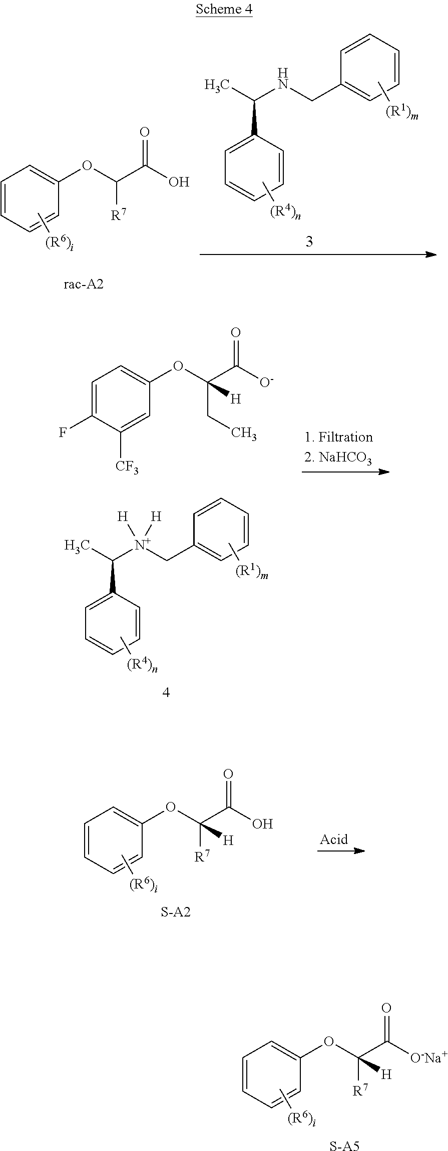

One can also appreciate that the procedures summarized in Schemes 2 and 4 can be used to obtain compounds of R-A2 or specifically Formula R-2, if desired, with equal efficiency if the R-enantiomer of a compound of Formula 3 is used.

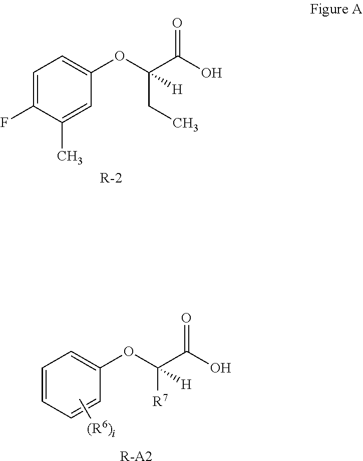

For industrial applicability and avoidance of waste, it is preferred that the undesired enantiomer in a resolution can be recycled to racemic material to be reused to prepare the desired enantiomer. This can be accomplished as summarized in Schemes 5 and 5A. Treating the mother liquors and washes obtained from the filtration of the solid product R,S-diastereomeric salt of Formula A4 can be treated as described in reference to Scheme 4 to obtain a scalemic mixture of Formula scal-A2 that is predominantly the R-2-aryloxyalkanoic acid. The compound of Formula scal-A2 can be converted to the corresponding acid chloride of Formula scal-A12, which can be treated with a tertiary amine to provide the compound of rac-A12 in essentially 0% ee. Suitable chlorinating agents include POCl₃, SOCl₂, (COCl)₂ or COCl₂. Thionyl chloride, SOCl₂, is a preferred chlorinating agent. Suitable solvents include acetonitrile, dichloroethane, toluene, tetrahydrofuran, dimethyl sulfoxide or N,N-dimethylformamide. Preferred solvents include N,N-dimethylformamide, dichloromethane, dichloroethane, toluene or acetonitrile, more preferably toluene. A preferred tertiary amine is triethylamine. Hydrolysis of the compound of Formula rac-A12 provides the compound of Formula rac-A2.

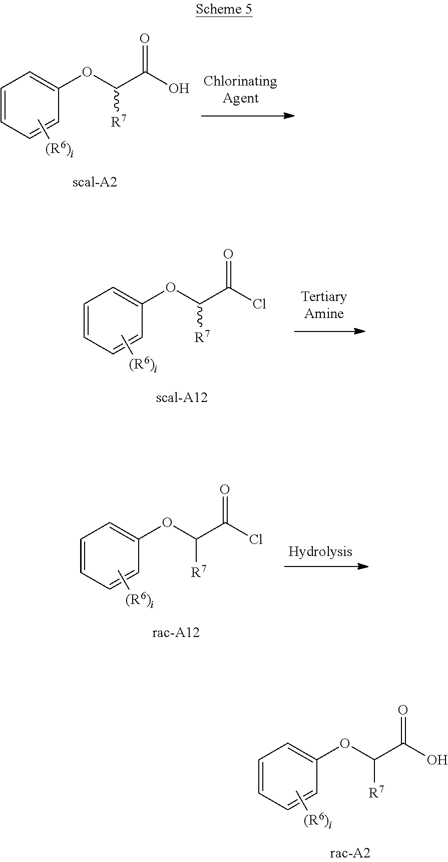

A specific example of the method shown in Scheme 5 above is shown in Scheme 5A below, wherein a compound of Formula scal-2 is racemized to the compound of rac-2.

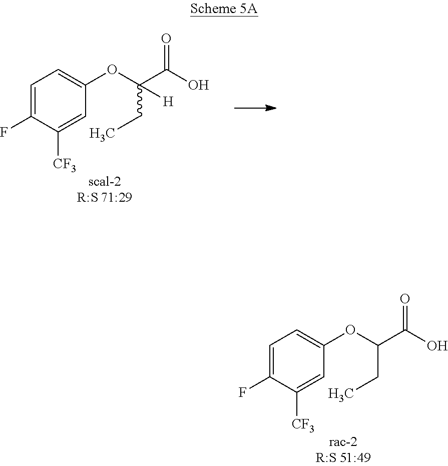 [SMILES: CCC(Oc1ccc(F)c(C(F)(F)F)c1)C(=O)O ; CCC(Oc1ccc(F)c(C(F)(F)F)c1)C(=O)O]

As shown in Scheme 6, compound S-2, prepared as in Scheme 2, can be converted to compound S-1 by treatment with a chlorinating agent to prepare compound S-12 followed by treatment with compound 7 (i.e. benzyl amine). Suitable chlorinating agents include POCl₃, SOCl₂, (COCl)₂ or COCl₂. Thionyl chloride, SOCl₂, is a preferred chlorinating agent.

Suitable solvents include acetonitrile, dichloroethane, toluene, tetrahydrofuran, dimethyl sulfoxide or N,N-dimethylformamide. Preferred solvents include N,N-dimethylformamide, dichloroethane, toluene or acetonitrile, more preferably toluene.

Compound S-6 can be treated with compound 7, optionally in the presence of an additional base, to provide compound S-1. Suitable solvents include acetonitrile, dichloromethane, dichloroethane, toluene, tetrahydrofuran, dimethyl sulfoxide or N,N-dimethylformamide. Preferred solvents include dichloromethane, dichloroethane, toluene or acetonitrile, more preferably dichloromethane or toluene, most preferably dichloromethane. Suitable additional bases for the reaction include alkali metal hydrides such as sodium hydride; or alkali metal alkoxides such as sodium isopropoxide and potassium tert-butoxide; or alkali metal hydroxides such as potassium hydroxide and sodium hydroxide; or alkali metal carbonates and bicarbonates such as sodium bicarbonate, potassium bicarbonate, sodium carbonate, potassium carbonate and cesium carbonate; or bases such as lithium bis(trimethylsilyl)amide, sodium bis(trimethylsilyl)amide and lithium di isopropylamide; or tertiary amines such as triethylamine and di isopropylethylamine. Preferred bases include triethylamine, sodium hydroxide, potassium hydroxide, sodium bicarbonate, potassium bicarbonate, sodium carbonate or potassium carbonate, preferably triethylamine.

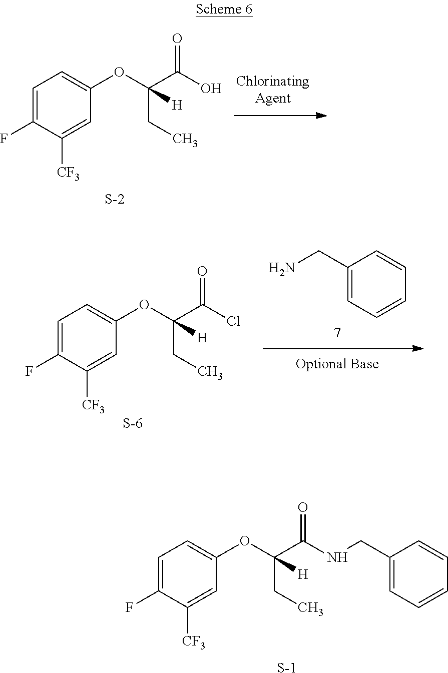

Alternatively, compound S-2 can be treated with compound 7 to prepare compound S-1. Preferably, the treatment comprises heating compound S-2 with about 2 to 5 molar equivalents of compound 7, such as about three equivalents, at about 100 to 125° C., such as about 110 to 120° C. Optionally, a solvent such as toluene can be used. The crude material obtained after removal of excess benzyl amine can be recrystallized from a mixture of isopropanol and water to provide the compound of Formula S-1.

In some embodiments, each of the compounds of Formulae rac-10 and compounds rac-2, S-2, S-6 can be isolated after preparation and before being carried into the next step. Alternatively, two or more of the steps from the compound of Formula rac-8 to compound S-1 can be combined without isolating the intermediate compound(s). For example, compound rac-2 can be prepared from the compound of Formula rac-8 without isolating the compound of Formula rac-10. In other embodiments, if compound S-2 is extracted with toluene from the aqueous phase after acidification of S-5, it can be treated with a chlorinating agent without isolation to prepare compound S-6. In other embodiments, conversion of compound S-2 to compound S-1 can be carried out without isolating compound S-6. In other embodiments, compound S-5 can be converted to compound S-1 without isolating compounds S-2 or S-6.

It is recognized that some reagents and reaction conditions described above for preparing compounds of Formulae 1-11 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after the introduction of a given reagent as it is depicted in any individual scheme, it may be necessary to perform additional routine synthetic steps not described in detail to complete the synthesis of compounds of Formulae 1-11. One skilled in the art will also recognize that it may be necessary to perform a combination of the steps illustrated in the above schemes in an order other than that implied by the particular sequence presented to prepare the compounds of Formulae 1-11. One skilled in the art will also recognize that compounds of Formulae 1-11 and the intermediates described herein can be subjected to various electrophilic, nucleophilic, radical, organometallic, oxidation, and reduction reactions to add substituents or modify existing substituents.

Without further elaboration, it is believed that one skilled in the art using the preceding description can utilize the present invention to its fullest extent. The following Examples are, therefore, to be construed as merely illustrative and not limiting of the disclosure in any way whatsoever. Steps in the following Examples illustrate a procedure for each step in an overall synthetic transformation, and the starting material for each step may not have necessarily been prepared by a particular preparative run whose procedure is described in other Examples or Steps. Percentages are by weight. The abbreviation “h” stands for “hour” or “hours”. The abbreviation “GCA” stands for “gas chromatographic analysis”.

### Synthesis Example 1

Step 1: Preparation of 2,4-dichloro-N-[(1R)-1-phenylethyl]-benzenemethanamine (CAS 1409308-40-2)

To a two-liter round bottomed flask fitted with stirrer, condenser and thermometer pocket were charged N,N-dimethylformamide (535 g), (R)-1-phenylethanamine (122.20 g, 1 mol), and potassium carbonate (202 g, 1.43 mol). To this mixture, 2,4-dichlorobenzyl chloride (188 g, 0.9557 mol) was added slowly at 28° C. The resulting slurry was heated to 45-46° C. and maintained at that temperature for 7 h. The reaction mass was cooled to 27-25 28° C. and the solids were removed by filtration and washed with N,N-dimethylformamide (2×105 g). N,N-Dimethylformamide was removed from the filtrate by distillation under reduced pressure. Heptanes (200 g) were added to the residue and the solids were removed by filtration. Heptane removal from the filtrate provided 268.9 g of the title compound as. Purity by GCA was 95.9% and yield was 92.0%.

### Synthesis Example 2

Resolution of racemic 2-(4-fluoro-3-(trifluoromethyl)phenoxy)butanoic acid

Step 1: Preparation of the salt of 2,4-dichloro-N-[(1R)-1-phenylethyl]-benzenemethanamine and (S)-2-(4-fluoro-3-(trifluoromethyl)phenoxy)butanoic acid.

To a five-liter round bottomed flask fitted with stirrer, condenser and thermometer pocket were charged solid racemic 2-(4-fluoro-3-(trifluoromethyl)phenoxy)butanoic acid (269.5 g, 1.0 mol) and heptane (2000 g). The resulting mixture was heated to 40° C. until a clear solution was obtained. The solution was cooled to 27° C. To this solution, a solution of 2,4-dichloro-N-[(1R)-1-phenylethyl]-benzenemethanamine) the title compound of Synthesis Example 1, 280.18 g, 1.0 mol) in heptane (500 g) was added slowly over 30 minutes at about 27 to 30° C. Stirring was continued for 1 h. The resulting slurry was heated to 65° C. to provide a clear solution. The solution was cooled to 50° C. and stirred for 0.5 h. The resulting slurry was cooled to about 30° C. and filtered. The filter cake was washed with heptane (2×400 g). The title diastereomeric salt was obtained as an off-white solid (245.5 g), in a yield of 45%. The salt was used in the next step without further purification.

Step 2: Preparation of (S)-2-(4-fluoro-3-(trifluoromethyl)phenoxy)butanoic acid.

To a two-liter round bottomed flask fitted with stirrer, condenser and thermometer pocket was charged the title salt of Synthesis Example 2, Step 1 (245.5 g 0.45 mol). Aqueous 10% NaOH (480 g, 1.2 mol) was added slowly at about 25 to 30° C. The pH of the reaction mass was maintained at 11 to 12° C. Toluene (800 g) was added and the resulting mixture was stirred for 0.5 h at 40° C. The phases were separated and the aqueous layer was extracted with toluene. The aqueous layer was acidified with hydrochloric acid (34%, 140.0 g, 1.4 mol) at about 25° C. Toluene was added and the resulting mixture was stirred for 0.5 h. The organic layer was separated and concentrated to dryness to provide the title compound (124 g) as an off-white solid, with purity by GCA of 98.1% and yield of 45.1% from the racemic acid; R:S ratio 2:98; ee 96%).

### Synthesis Example 3

Racemization of Scalemic 2-(4-fluoro-3-(trifluoromethyl)phenoxy)butanoic acid

Step 1: Preparation of scalemic 2-(4-fluoro-3-(trifluoromethyl)phenoxy)butanoic acid chloride.

The combined mother liquors and washings obtained from the filtration of the solid product according to Step 1, Synthesis Example 2 were treated according to the procedure of Step 2, Synthesis Example 2 to recover a scalemic mixture of 79% (S)-2-(4-fluoro-3-(trifluoromethyl)phenoxy)butanoic acid and 21% (R)-2-(4-fluoro-3-(trifluoromethyl)phenoxy)butanoic acid (58% ee).

To a one-liter round bottomed flask fitted with stirrer, condenser, thermometer pocket, and gas scrubbers, was charged scalemic 2-(4-fluoro-3-(trifluoromethyl)phenoxy)butanoic acid (273.35 g 1 mol). Thionyl chloride (240.80 g 2.0 mol) was added over 1 h at 26-30° C. The mixture was heated to 70° C. and maintained at that temperature for 2 h. Vent gases were scrubbed into water followed by a dilute NaOH aqueous solution scrubber. The reaction was monitored by GC for completion. Excess thionyl chloride was removed by distillation under reduced pressure. The title compound (278.0 g) (R:S ratio of 71:29) was obtained as an oily residue and it was used in the next step without purification.

Step 2: Preparation of racemic 2-(4-fluoro-3-(trifluoromethyl)phenoxy)butanoic acid.

To a three-liter round bottomed flask fitted with stirrer, condenser and thermometer pocket was charged the title compound of Synthesis Example 3, Step 1, (287.50 g, 1 mol) and toluene (1200 g), then cooled to 0-3° C. Triethyl amine (204.50 g, 2 mol) was added to obtain a slurry. The reaction mixture was maintained at 28° C. for 7 h. GC monitoring showed the R:S ratio to be 50.5:49.5. The reaction mass was subjected to hydrolysis with aqueous sodium hydroxide, 10% solution, (802.4 g, 2.06 mol) by stirring at 70° C. for 3 h. The reaction product was extracted with toluene (600 g). The aqueous layer was acidified using hydrochloric acid and then extracted with toluene twice (1500 g, then 500 g). The combined organic layers were washed with water (500 g) and concentrated to obtain 238 g of the title compound (Purity by GC analysis: 97.42%, Yield: 81.5%, Chiral ratio by GCA was an R:S ratio of 51:49).

### Synthesis Example 4

Preparation of (S)-N-benzyl-2-(4-fluoro-3-trifluoromethylphenoxy)-butanoic amide

Step 1: Preparation of (S)-2-(4-fluoro-3-(trifluoromethyl)phenoxy)butanoic acid chloride.

To a reactor stirrer, condenser and thermometer pocket was charged (S)-2-(4-fluoro-3-(trifluoromethyl)phenoxy)butanoic acid (about 385 mmol), i.e. the title material from Synthesis Example 2, Step 2, and dichloromethane (177 g) was added. A caustic scrubber was attached and pyridine (0.9 g, 11 mmol) was added. Thionyl chloride (52.1 g, 438 mmol) was added over about 10 minutes, with evolution of white fumes. The reactor jacket was adjusted to about 50-55° C. so that the reaction mass was brought to reflux at about 40° C. Sulfur dioxide and hydrogen chloride off-gassing was routed through the caustic scrubber. The reflux was continued for about 3 to 5 h, until the amount of the starting acid was less than about 0.5% by GC analysis of an aliquot. The pressure in the reactor was reduced to about 110 mbar and the volatiles were removed by distillation. The pressure was adjusted to 10 mbar to ensure complete removal of all volatiles. The reactor was brought to ambient temperature and pressure under nitrogen and the reactor was fitted with a calcium chloride-packed drying tube to provide the title compound. The crude material was used directly in the next step.

Step 2: Preparation of (S)-N-benzyl-2-(4-fluoro-3-trifluoromethylphenoxy)-butanoic amide

To the reactor containing (S)-2-(4-fluoro-3-(trifluoromethyl)phenoxy)butanoic acid chloride (about 385 mmol), i.e. the title material from Synthesis Example 4, Step 1, was added dichloromethane (177 g). The solution was charged to a jacketed reactor, fitted with a mechanical stirrer, thermometer and a dropping funnel and the reaction mixture was cooled to about 5° C. A mixture of benzylamine (43.2 g, 438 mmol) and triethylamine (44.3, 438 mmol) was added via the dropping funnel over a period of about 1 h while maintaining the temperature below 10° C. The reaction during addition was highly exothermic. After the addition was complete, the reaction mixture was stirred for about 30 minutes, until the amount of the starting acid chloride was less than about 0.5% by GC analysis of an aliquot. The temperature was adjusted to about 5° C. and water (131.8) was added. After 10 minutes, the phases were separated and the aqueous layer was removed. The volatiles, mostly dichloromethane, were removed by lowering the pressure to about 100 mbar and then incrementally increasing the reaction mass temperature to 70° C. to keep the reaction mixture molten. Isopropanol (200 g) was added and the resulting mixture was cooled to about 40° C. and stirred for about 15 minutes. Water (90.1 g) was added to provide a mixture that corresponds to a solution of the title compound (about 30% w/w) in an isopropanol:water mixture of 7:3. The solution was seeded with (S)-N-benzyl-2-(4-fluoro-3-trifluoromethylphenoxy)-butanoic amide (2 g, 2% seeding amount) from a previous run and then cooled using the following cooling profile

| Time (h) | Temperature (° C.) |
|---|---|
| 0 | 40 |
| 20 | 25 |
| 24 | 5. |

The resulting slurry was filtered at 5° C. and the cold filtrate was used to wash the reactor and the filter cake. The filter cake was not washed any further to avoid re-dissolving the product. The filter cake was dried on the filter under suction and further air-dried to provide the title compound with a purity of about 95%. b 2

What is claimed is:

1. A method for preparing compound S-1 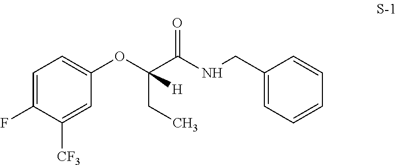 [SMILES: CC[C@H](Oc1ccc(F)c(C(F)(F)F)c1)C(=O)NCc1ccccc1]
from compound S-2 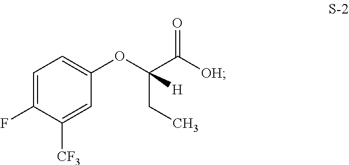 [SMILES: CC[C@H](Oc1ccc(F)c(C(F)(F)F)c1)C(=O)O]
wherein compound S-2 is prepared by treating compound rac-2 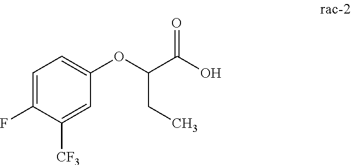 [SMILES: CCC(Oc1ccc(F)c(C(F)(F)F)c1)C(=O)O]
with a compound of Formula 3 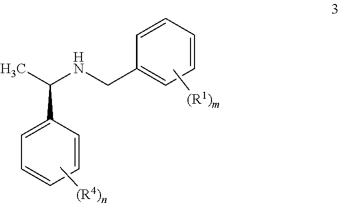
wherein
each R¹ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R²; or
two adjacent R¹ substituents are taken together with the phenyl to which they are attached to form a naphthalenyl ring optionally substituted with up to three R³;
each R² and each R³ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
each R⁴ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R⁵;
each R⁵ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
m is 0, 1, 2 or 3; and
n is 0, 1, 2 or 3;
to provide a R,S-salt of Formula 4 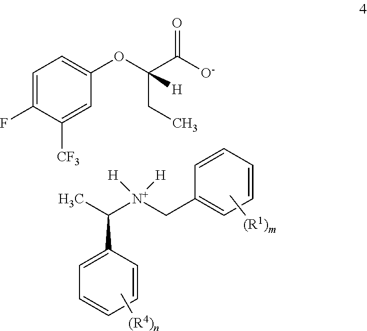
wherein R¹, R⁴, m and n are as defined above;
selectively isolating the R,S-salt of Formula 4;
treating the R,S-salt of Formula 4 with a sodium base to provide carboxylate salt S-5 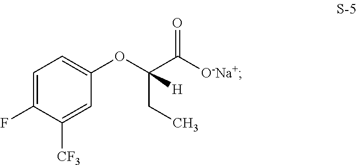
and
treating carboxylate salt S-5 with acid.

2. The method of claim 1 wherein
m is 1 or 2;
n is 0; and
each R¹ is independently halogen, nitro, C₁-C₄ alkyl, C₁-C₄ haloalkyl; or phenyl; or
two adjacent R¹ substituents are taken together with the phenyl to which they are attached to form a naphthalenyl ring optionally substituted with up to two R³.

3. The method of claim 2 wherein each R¹ is independently halogen, nitro, C₁-C₄ alkyl or C₁-C₄ haloalkyl.

4. The method of claim 1 wherein the compound of Formula 3 is selected from the group consisting of
(αR)-α-methyl-N-(phenylmethyl)-benzenemethanamine,
N-[(1R)-1-phenylethyl]-1-naphthalenemethanamine,
2,4-dichloro-N-[(1R)-1-phenylethyl]-benzenemethanamine,
3,4-dichloro-N-[(1R)-1-phenylethyl]-benzenemethanamine,
2,6-dichloro-N-[(1R)-1-phenylethyl]-benzenemethanamine,
2,4,6-trimethyl-N-[(1R)-1-phenylethyl]-benzenemethanamine,
4-nitro-N-[(1R)-1-phenylethyl]-benzenemethanamine, and
2-methyl-3-phenyl-N-[(1R)-1-phenylethyl]-benzenemethanamine.

5. The method of claim 1 further comprising
treating compound S-2 with a chlorinating agent to prepare compound S-6 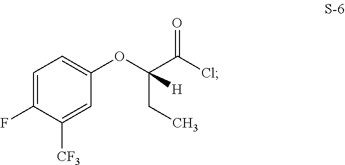 [SMILES: CC[C@H](Oc1ccc(F)c(C(F)(F)F)c1)C(=O)Cl]
treating compound S-6 with compound 7 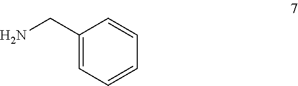
to prepare compound S-1.

6. The method of claim 1 wherein compound rac-2 is prepared by treating a compound of Formula rac-8 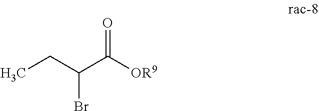
wherein R⁹ is C₁-C₆ alkyl;
with compound 9 in the presence of a base 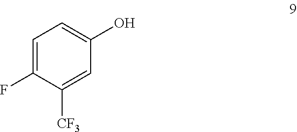 [SMILES: Oc1ccc(F)c(C(F)(F)F)c1]
to provide a compound of Formula rac-10 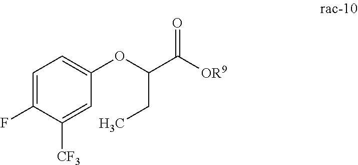
wherein R⁹ is C₁-C₆ alkyl; and
hydrolyzing the compound of Formula rac-10.

7. A method for preparing compound S-1 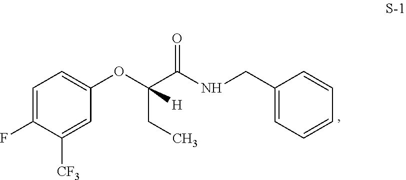 [SMILES: CC[C@H](Oc1ccc(F)c(C(F)(F)F)c1)C(=O)NCc1ccccc1]
the method comprising:
treating compound rac-2 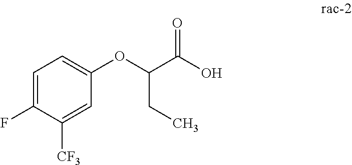 [SMILES: CCC(Oc1ccc(F)c(C(F)(F)F)c1)C(=O)O]
with a compound of Formula 3 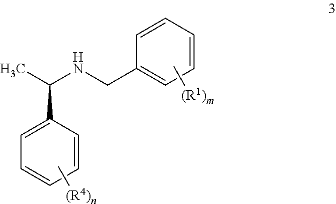
wherein
each R¹ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R²; or
two adjacent R¹ substituents are taken together with the phenyl to which they are attached to form a naphthalenyl ring optionally substituted with up to three R³;
each R² and each R³ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
each R⁴ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R⁵;
each R⁵ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
m is 0, 1, 2 or 3; and
n is 0, 1, 2 or 3;
to provide the R,S-salt of Formula 4 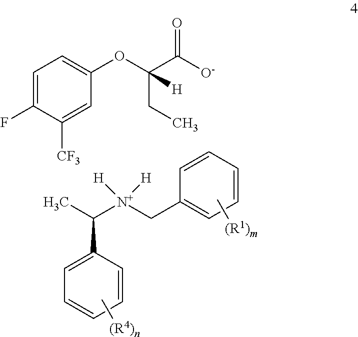
wherein R¹, R², R³, R⁴, m and n are as defined above;
selectively isolating the R,S-salt of Formula 4;
treating the R,S-salt of Formula 4 with a sodium base to provide carboxylate salt S-5 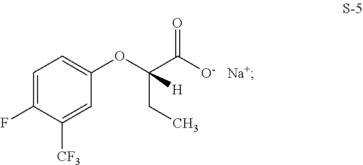
treating the carboxylate salt S-5 with acid to prepare compound S-2 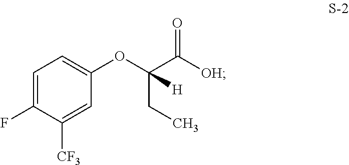 [SMILES: CC[C@H](Oc1ccc(F)c(C(F)(F)F)c1)C(=O)O]
converting compound S-2 to compound S-1.

8. The method of claim 7 wherein
m is 1 or 2;
n is 0; and
each R¹ is independently halogen, nitro, C₁-C₄ alkyl, C₁-C₄ haloalkyl; or phenyl; or
two adjacent R¹ substituents are taken together with the phenyl to which they are attached to form a naphthalenyl ring optionally substituted with up to two R³.

9. The method of claim 8 wherein each R¹ is independently halogen, nitro, C₁-C₄ alkyl or C₁-C₄ haloalkyl.

10. The method of claim 8 wherein the compound of Formula 3 is selected from the group consisting of
(αR)-α-methyl-N-(phenylmethyl)-benzenemethanamine,
N-[(1R)-1-phenylethyl]-1-naphthalenemethanamine,
2,4-dichloro-N-[(1R)-1-phenylethyl]-benzenemethanamine,
3,4-dichloro-N-[(1R)-1-phenylethyl]-benzenemethanamine,
2,6-dichloro-N-[(1R)-1-phenylethyl]-benzenemethanamine,
2,4,6-trimethyl-N-[(1R)-1-phenylethyl]-benzenemethanamine,
4-nitro-N-[(1R)-1-phenylethyl]-benzenemethanamine, and
2-methyl-3-phenyl-N-[(1R)-1-phenylethyl]-benzenemethanamine.

11. The method of claim 8 wherein converting compound S-2 to compound S-1 comprises
treating compound S-2 with a chlorinating agent to prepare compound S-6 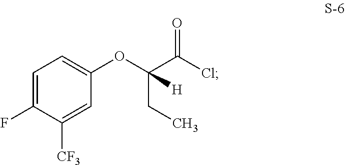 [SMILES: CC[C@H](Oc1ccc(F)c(C(F)(F)F)c1)C(=O)Cl]
treating compound S-6 with compound 7

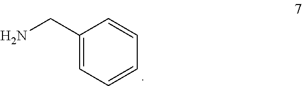

12. The method of claim 8 wherein compound rac-2 is prepared by treating a compound of Formula rac-8 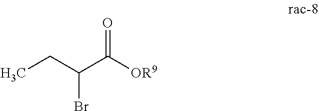
wherein R⁹ is C₁-C₆ alkyl;
with compound 9 in the presence of a base 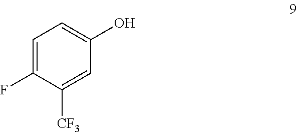 [SMILES: Oc1ccc(F)c(C(F)(F)F)c1]
to provide a compound of Formula rac-10 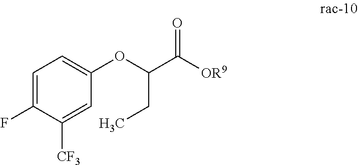
wherein R⁹ is C₁-C₆ alkyl; and
hydrolyzing the compound of Formula rac-10.

13-23. (canceled)

24. An R,S-salt of Formula A4 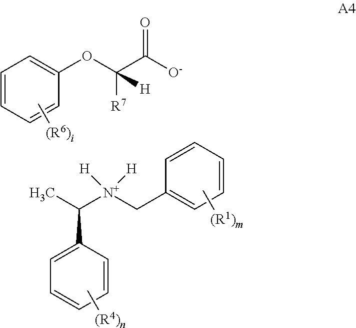
wherein
each R¹ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R²; or
two adjacent R¹ substituents are taken together with the phenyl to which they are attached to form a naphthalenyl ring optionally substituted with up to three R³;
each R² and each R³ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
each R⁴ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R⁵;
each R⁵ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
each R⁶ is independently halogen, nitro, cyano, C₁-C₄ alkyl, C₁-C₄ haloalkyl, C₁-C₄ alkoxy, C₁-C₄ alkenyl, C₁-C₄ haloalkenyl or C₁-C₄ haloalkoxy;
R⁷ is C₁-C₆ alkyl, C₁-C₆ haloalkyl, C₁-C₆ alkenyl or C₁-C₆ haloalkenyl;
i is 0, 1,2 or 3;
m is 0, 1, 2 or 3; and
n is 0, 1,2 or 3.

25. The R,S-salt of claim 24 wherein each R¹ is independently halogen, nitro, C₁-C₄ alkyl or C₁-C₄ haloalkyl.

26. The R,S-salt of claim 24 that is a salt of an amine selected from the group consisting of
(αR)-α-methyl-N-(phenylmethyl)-benzenemethanamine,
N-[(1R)-1-phenylethyl]-1-naphthalenemethanamine,
2,4-dichloro-N-[(1R)-1-phenylethyl]-benzenemethanamine,
3,4-dichloro-N-[(1R)-1-phenylethyl]-benzenemethanamine,
2,6-dichloro-N-[(1R)-1-phenylethyl]-benzenemethanamine,
2,4,6-trimethyl-N-[(1R)-1-phenylethyl]-benzenemethanamine,
4-nitro-N-[(1R)-1-phenylethyl]-benzenemethanamine, and
2-methyl-3-phenyl-N-[(1R)-1-phenylethyl]-benzenemethanamine.

27. The R,S-salt of claim 24 wherein m is 2 and the R¹ substituents are chloro at the 2— and 4-positions; and n is 0.

28. The R,S-salt of claim 24 wherein each R⁶ is independently halogen, C₁-C₄ alkyl, C₁-C₄ alkoxy or C₁-C₄ haloalkyl; and
R⁷ is C₁-C₆ alkyl.

29. The R,S-salt of claim 24 wherein each R⁶ is independently halogen, C₁-C₄ alkyl or C₁-C₄ haloalkyl; and
R⁷ is C₁-C₂ alkyl.

30. The R,S-salt of claim 24 wherein i is 2; one R⁶ is 3-CF₃, the second R⁶ is 4-F and R⁷ is ethyl.

31. The R,S-salt of claim 24 wherein
m is 2 and the R¹ substituents are chloro in the 2— and 4-positions;
n is 0;
i is 2;
one R⁶ is 3-CF₃ and the second R⁶ is 4-F; and
R⁷ is ethyl.
